(19) United States
(12) Patent Application Publication
(10) Pub. No.: US 2004/0006313 A1
(43) Pub. Date: Jan. 8, 2004

(54) Retractable safety syringe cylinder

(76) Inventor: Lai-Wen Chian, Taipei (TW)

Correspondence Address: Lai-Wen Chian, 235 Chung- Ho, Box 8-24, Taipei, TW

(21) Appl. No.: 10/189,325

(22) Filed: Jul. 8, 2002

(51) Int. Cl. A61M 5/32

(52) U.S. Cl. 604/198

(57) ABSTRACT

A retractable safety syringe cylinder has a structure which precisely hooks the syringe needle for retracting the syringe in the syringe cylinder for bending A hole seat at a lower end of the syringe needle is matched to a hook body at a top surface of a rod cap of a push rod. After pushing injection liquid, the two are engaged so as to control the displacement of the syringe needle. Thus, the waste from the syringe is controllable and the operation is convenient.

### FIELD OF THE INVENTION

The present invention relates to medical devices, and particularly to a retractable safety syringe cylinder which has a structure capable of precisely hooking the syringe needle for retracting the syringe easily so as to control the displacement of the syringe needle. Thus, the waste from the syringe is controllable and the operation is convenient.

### BACKGROUND OF THE INVENTION

Subcutaneous injections are frequently used in the medical treatment. Retractable safety syringe injectors are especially suitable for subcutaneous injection, which has the following advantages:

1. The use of the retractable safety syringe cylinder is similar to the conventional injector, it can be operated by a simple training.

2. The retractable safety syringe cylinder is safe and retractable so as not to hurt anybody.

3. It is disposable after used once.

FIG. 7 shows the structure of a retractable safety syringe cylinder. It contains a syringe cylinder 1, a syringe needle 2, a push rod 3, and others. The upper end of the syringe cylinder 1 has a syringe connecting seat 11 having a positioning hole 110 therein. The hole has a positioning groove 111 for retaining the syringe needle 2. The push rod 3 has a rod cap 30 which has a hook body 301. The needle of the syringe needle 2 has a slot 201. A bottom of the slot 201 is inclined. A periphery of the needle has a ring retained by the positioning groove 111.

The use of the retractable safety syringe cylinder will be described herein. At first, the injector is checked. After injection, the hook body 301 can be inserted to the slot 201 correctly. The syringe needle 2 is exactly combined to the hook body 301 at a top of the push rod 3. when the push rod 3 is pulled backwards, the syringe needle 2 will retract into the syringe cylinder 1. Since the bottom of the slot 201 is inclined, after the hook body 301 of the push rod 3 is inserted. The syringe needle 2 is inclined in the syringe cylinder 1, as shown in the FIG. 8, so as to prevent from hurting anybody as it is deserted.

Although this retractable safety syringe cylinder has many advantages, when the push rod 3 is pulled backwards, it often becomes loose and thus the hook body 301 can not be inserted into the slot 201 so that it can not be processed conveniently.

### SUMMARY OF THE INVENTION

Accordingly, the primary object of the present invention is to provide a retractable safety syringe cylinder which has a firm and security structure.

To achieve above objects, the present invention provides a retractable safety syringe cylinder has a structure which precisely hook the syringe needle for retracting the syringe in the syringe cylinder for bending. A hole seat at a lower end of the syringe needle is matched to a hook body at a top surface of a rod cap of a push rod. After pushing injection liquid, the two are engaged so as to control the displacement of the syringe needle. Thus, the waste from the syringe is controllable and the operation is convenient.

The various objects and advantages of the present invention will be more readily understood from the following detailed description when read in conjunction with the appended drawing.

### BRIEF DESCRIPTION OF THE DRAWINGS

FIG. 1 is a structural schematic view of the present invention.

FIG. 2 is an exploded perspective view of the present invention.

FIG. 3 is a partial structural view of the present invention.

FIG. 4 shows one embodiment of the present invention.

FIG. 5 is a structural cross section view of the present invention.

FIG. 6 shows one embodiment of the present invention.

FIG. 7 is a structural schematic view of one prior art.

FIG. 8 is a partial structural schematic view of the prior art.

### DETAILED DESCRIPTION OF THE PREFERRED EMBODIMENTS

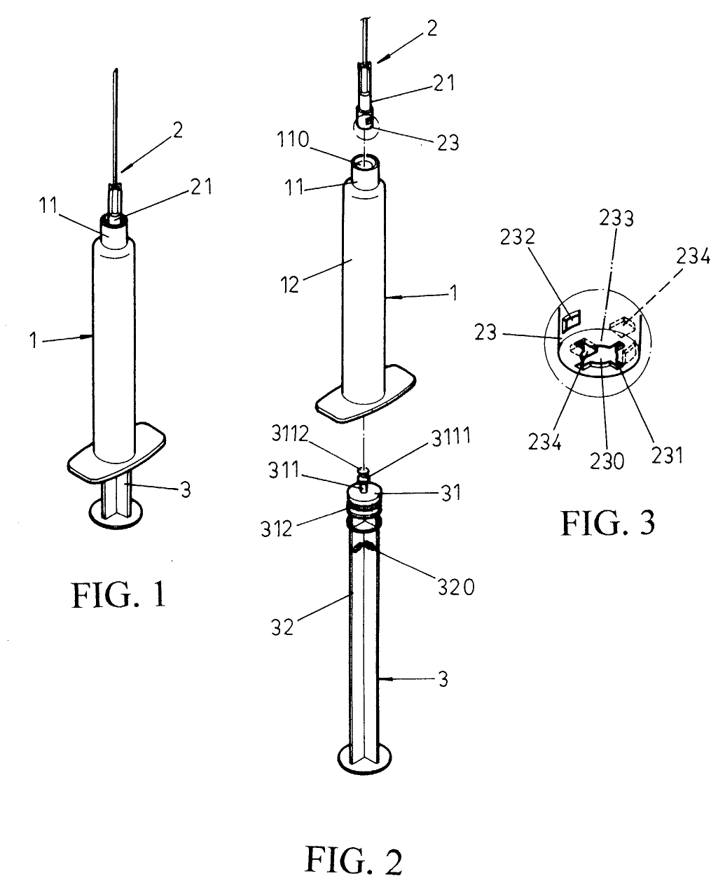

Referring to FIGS. 1 and 2, the retractable safety syringe cylinder of the present invention is illustrated. The retractable safety syringe cylinder includes a syringe cylinder 1, a syringe needle 2, and a push rod 3.

The upper center of the syringe cylinder 1 has a syringe connecting seat 11. The syringe connecting seat 11 has a positioning hole 110. The lower end of the cylinder body 12 has a ring 121.

The upper end of the syringe needle 2 is a needle tube 22 and the lower end is a needle 21. A lower end of the needle 21 has a hole seat 23. A lower end of the hole seat 23 is a slot 231. A hook chamber 233 is adjacent to the slot 231. The hook chamber 233 has two transversal hook blocks 234. A center of the hole seat 23 is a channel hole 230. The channel hole 230 penetrates through the needle body so as to communicate to the needle tube 22. The hole seat 23 is installed with a guide hole 232, as shown in the FIG. 3.

A top of the push rod 3 is a rod cap 31. The rod cap 31 can be made of elastic material. A periphery of the rod cap 31 has a positioning groove 312. A top center of the rod cap 31 has a hook body 311. The hook body 311 has two wedge buckles 3111 and 3112 which are formed as two stages. The upper wedge buckles 3112 is slightly smaller than the lower wedge buckle 3111. The rod body 32 has a through hole 320, as shown in the FIG. 2.

Thereby, the syringe needle 2 can pass through the positioning hole 110 of the syringe connecting seat 11 of the syringe cylinder 1. The guide hole 232 in the hole seat 23 of the syringe needle 2 is helpful to guide the injection liquid.

Thereby, the inner side of the cylinder body 12 of the syringe cylinder 1 has a ring 121 which can be engaged to the positioning groove 312 installed at the rod cap 3 1 of the push rod 3 so as to prevent the push rod 3 from trapping in the cylinder body 12 as the push rod 3 move outwards.

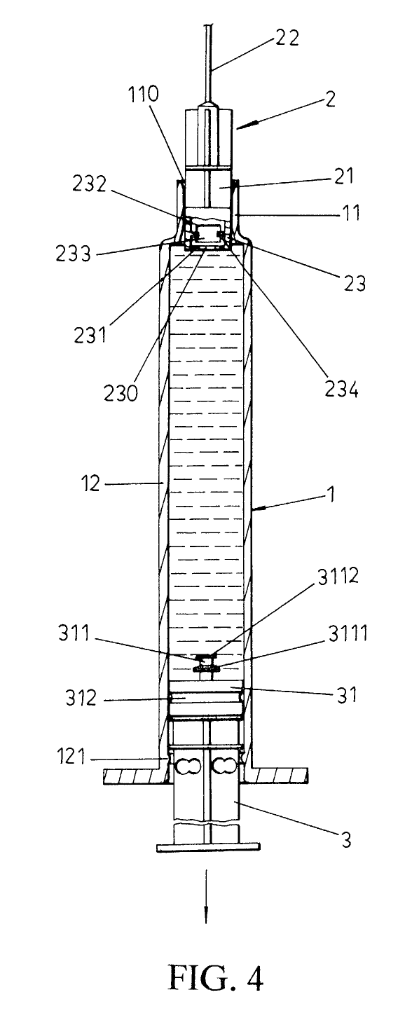

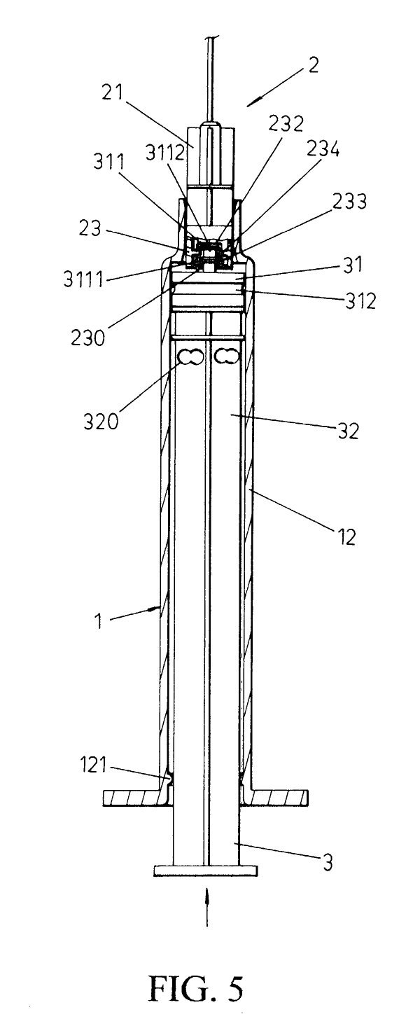

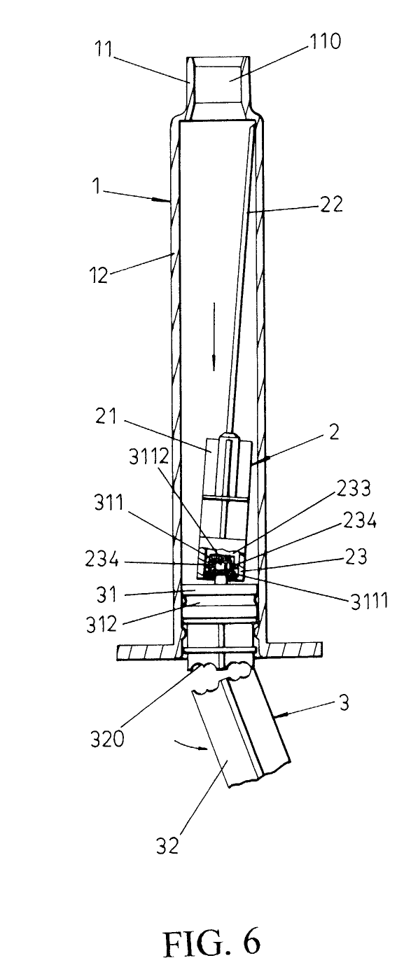

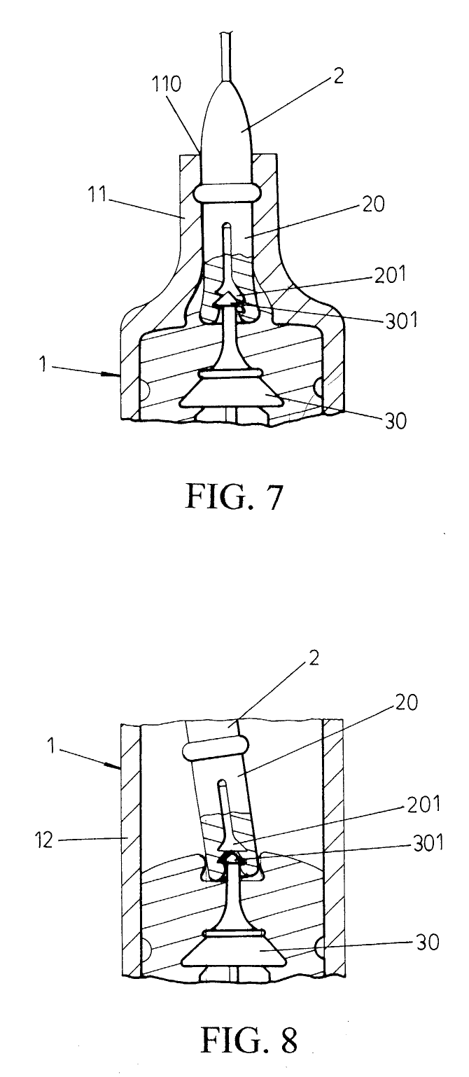

By above construction, the retractable safety syringe cylinder can be formed. The retractable safety syringe cylinder of the present invention is used once for preventing from being infected. Referring to FIG. 4, the syringe cylinder 1 is full of injection liquid. By pushing the push rod 3, the liquid will flow through the channel hole 230 of the needle 21 and then is sent out form the syringe tube 22. By further pushing the push rod 3, after all the liquid has been sent out, the rod cap 31 at the top of the push rod 3 exactly resists against the hole seat 23 of the needle 21. Since the needle 21 has-the hole seat 23, the hook body 311 on the rod cap 31 exactly inserts into the slot 231 to enter into the hook chamber 233 and is buckled to the hook blocks 234 in the hook chamber 233, as shown in the FIG. 5. When the push rod 3 moves back again, the syringe needle 2 will retract from the syringe connecting seat II to be within the cylinder body 12, as shown in the FIG. 6. When the hook body 311 inserts into the hole seat 23, since the space of the hook chamber 233 is slightly larger than the hook body 311. The hook chamber 233 is loosely engaged to the wedge buckles 3111 and 3112. The wedge buckle 3112 is confined by the hook block 234 and thus can not retract out. Thus, although the syringe needle 2 can tilt and shift, it can not separated from the cylinder body 12 and thus is left therein. By installing the through hole 320, the rod body 32 can be broken easily. The operator will not be hurt.

Although the present invention has been described with reference to the preferred embodiments, it will be understood that the invention is not limited to the details described thereof. Various substitutions and modifications have been suggested in the foregoing description, and others will occur to those of ordinary skill in the art. Therefore, all such substitutions and modifications are intended to be embraced within the scope of the invention as defined in the appended claims.

What is claimed is:

1. A retractable safety syringe cylinder comprising a syringe cylinder, a syringe needle, and a push rod; characterized in that:
an upper end of the syringe cylinder has a syringe connecting seat and the syringe connecting seat has a positioning hole at a center thereof;
an upper end of the syringe needle is a needle tube and the lower end thereof is a needle; a lower end of the needle has a hole seat; a lower end of the hole seat is a slot; a hook chamber is adjacent to the slot; the volume of the hook chamber is slightly larger than that of the hook body; the hook chamber has two transversal hook blocks; a center of the hole seat is a channel hole; a channel hole penetrates through the needle body so as to communicate to the needle tube; and
a top of the push rod has a hook body; a hook body has two wedge buckles which are formed as a two stage structure;
thereby, the syringe needle passes through the positioning hole of the syringe connecting seat of the syringe cylinder; the guide hole in the hole seat of the syringe needle is helpful to guide the injection liquid.

2. The retractable safety syringe cylinder as claim in claim 1, wherein there are at least two hook blocks in the hook chamber.

3. The retractable safety syringe cylinder as claim in claim 1, wherein a lower end of the cylinder body has a ring which is matchable with a positioning groove in the rod cap of the push rod so as to confine the retracted range of the cylinder body.

4. The retractable safety syringe cylinder as claim in claim 1, wherein the hole seat of the syringe needle has a guide hole for guiding the flow of syringe liquid.

5. The retractable safety syringe cylinder as claim in claim 1, wherein the two wedge buckles are an upper wedge buckle and a lower exhaust tube; the upper wedge buckle is slightly smaller than the lower wedge buckle.
